(19) United States
(12) Patent Application Publication
(10) Pub. No.: US 2024/0122923 A1
(43) Pub. Date: Apr. 18, 2024

(54) METHODS OF TREATING HIDRADENITIS SUPPURATIVA

(71) Applicant: AbbVie Inc., North Chicago, IL (US)

(72) Inventors: Aileen L. PANGAN, La Grange, IL (US); Mohamed-Eslam F. Mohamed, Libertyville, IL (US)

(21) Appl. No.: 18/476,916

(22) Filed: Sep. 28, 2023

Related U.S. Application Data
(60) Provisional application No. 63/411,954, filed on Sep. 30, 2022.

(51) Int. Cl. A61K 31/4985; A61K 9/00; A61P 17/10

(52) U.S. Cl. CPC A61K 31/4985; A61K 9/0053; A61P 17/10

(57) ABSTRACT

The present disclosure is directed to methods for treating hidradenitis suppurativa (HS) using the selective JAK1 inhibitor upadacitinib.

### CROSS-REFERENCE TO RELATED APPLICATIONS

This application claims the benefit of U.S. Provisional Application No. 63/411,954, filed Sep. 30, 2022, and which is herein incorporated by reference in its entirety.

### FIELD OF THE DISCLOSURE

The present disclosure is directed to methods for treating hidradenitis suppurativa (HS) with the selective JAK1 inhibitor, upadacitinib.

### BACKGROUND OF THE DISCLOSURE

Hidradenitis suppurativa (HS) refers to a debilitating skin disorder of the apocrine glands (sweat glands found on certain parts of the body) and hair follicles in which swollen, painful, chronically inflamed lesions or lumps develop. HS is confined to areas of the body that contain apocrine glands, such as axillae, areola of the nipple, groin, perineum, circumanal, and periumbilical regions. It is speculated that immunological abnormalities of the hair follicle play a role in the etiology of this disease. HS is a recurring or chronic inflammatory condition affecting particularly young adults, with an average age of onset of 23 years. This poorly understood disease is believed to be under-reported by those who suffer from it but is estimated to affect approximately 1% of the general population in the West, with females affected 2 to 5 times more commonly than males (Naldi, L. Epidemiology; In Hidradenitis Suppurativa, Jemec et al., ed; Heidelberg: Springer 2006).

HS is characterized by recurrent inflamed nodules, abscesses, and fistulas, and occurs when apocrine gland outlets become blocked by perspiration or are unable to drain normally because of incomplete gland development. Secretions trapped in the glands force perspiration and bacteria into surrounding tissue, causing subcutaneous induration, inflammation, and infection. HS lesions (i.e., nodules, abscesses, and sinuses) are painful and can be malodorous with purulent discharge. This constellation of signs and symptoms results in substantial disability and social stigma for the patients and a profound impact on quality of life.

Current therapies for moderate to severe HS include short- or long-term oral or topical antibiotics, retinoids, intralesional steroids, oral steroids, immunosuppressive agents such as cyclosporine or methotrexate, radiation, laser therapy and tumor necrosis factor-α (TNF-α) antagonist adalimumab. However, adalimumab is the only approved treatment for HS, and other TNF antagonists, such as etanercept, have failed to show improvement of HS over a 24-week treatment period (Adams et al., Arch Dermatol. 2010, 146(5): 501-504). Given the limited success of treatments for HS and the debilitating nature of this disease, there is a pressing need for an effective treatment.

### SUMMARY OF THE DISCLOSURE

The present disclosure provides methods for treating hidradenitis suppurativa (HS) with the selective JAK1 inhibitor, upadacitinib.

In one aspect is provided a method for treating a human patient having moderate to severe hidradenitis suppurativa (HS), the method comprising orally administering once daily to the patient 30 mg of upadacitinib.

In some embodiments, the patient is an adult.

In some embodiments, the method comprises orally administering upadacitinib to the patient daily for up to 12 weeks. In some embodiments, the method comprises orally administering upadacitinib to the patient daily for at least 12 weeks. In some embodiments, the method comprises orally administering upadacitinib to the patient daily for at least 16 weeks. In some embodiments, the method comprises orally administering upadacitinib to the patient daily for up to 16 weeks, up to 20 weeks, up to 24 weeks, up to 28 weeks, up to 36 weeks, up to 44 weeks, up to 52 weeks, up to 64 weeks, up to 76 weeks, up to 88 weeks, up to 100 weeks, or up to 104 weeks.

In some embodiments, the number of inflammatory lesions (AN count) is decreased by at least 50% at 12 weeks after the first daily administration, relative to an AN count prior to initiating treatment.

In some embodiments, a reduction in the Pain Numeric Rating Scale 30 (PNRS30) is achieved at 12 weeks after the first daily administration.

In some embodiments, the patient achieves a Hidradenitis Suppurativa Clinical Response 50 (Hi SCR50) is achieved at 12 weeks after the first daily administration.

In some embodiments, the patient has had an inadequate response or intolerance to oral antibiotics.

In some embodiments, the patient has had an inadequate response or intolerance to anti-TNF therapy.

In some embodiments, the patient has HS lesions in at least two distinct anatomic areas prior to initiating treatment.

In some embodiments, the patient has a total AN count equal to or greater than 5 prior to initiating the treatment.

In some embodiments, the patient has a draining fistula count of less than or equal to 20 prior to initiating the treatment.

In some embodiments, the patient experiences a reduction in flare through week 12, defined as an increase in AN count of at least 25% with a minimum increase of 2 relative to baseline.

In some embodiments, the patient achieves, at 12 weeks after the first daily administration, relative to that in a patient who has not received the treatment, an improvement from baseline in Dermatology Life Quality Index (DLQI).

In some embodiments, the patient achieves, at 12 weeks after the first daily administration, relative to that in a patient who has not received the treatment, an improvement from baseline in Hidradenitis Suppurativa Symptom Assessment (HSSA) is achieved, relative to that in a patient who has not received the treatment.

In some embodiments, the patient achieves, at 12 weeks after the first daily administration, relative to that in a patient who has not received the treatment, an improvement from baseline in HS-related swelling, assessed based on the HSSA is achieved, relative to that in a patient who has not received the treatment.

In some embodiments, the patient achieves, at 12 weeks after the first daily administration, relative to that in a patient who has not received the treatment, an improvement from baseline in HS-related odor, assessed based on the HSSA is achieved, relative to that in a patient who has not received the treatment.

In some embodiments, the patient achieves, at 12 weeks after the first daily administration, relative to that in a patient who has not received the treatment, an improvement from baseline in HS-related worst drainage, assessed based on the HSSA, is achieved, relative to that in a patient who has not received the treatment.

In another aspect is provided a method for treating a human patient having moderate to severe hidradenitis suppurativa (HS), wherein the patient has failed to respond to or was intolerant of anti-TNF therapy, the method comprising orally administering once daily to the patient 30 mg of upadacitinib.

In some embodiments, the patient is at least 12 years of age. In some embodiments, the patient is an adult.

In some embodiments, the method comprises orally administering upadacitinib to the patient daily for at least 16 weeks.

In some embodiments, a Hidradenitis Suppurativa Clinical Response 50 (HiSCR 50) is achieved at Week 16. In some embodiments, a Hidradenitis Suppurativa Clinical Response 75 (HiSCR 75) is achieved at Week 16. In some embodiments, a Hidradenitis Suppurativa Clinical Response 90 (HiSCR 90) is achieved at Week 16.

In some embodiments, the number of inflammatory lesions (AN count) is decreased in the patient relative to an AN count prior to initiating treatment.

In some embodiments, a draining fistula count is decreased in the patient relative to a draining fistula count prior to initiating treatment.

In some embodiments, a Numerical Rating Scale 30 (NRS30) is achieved in a Patient's Global Assessment of HS-related skin pain at Week 2, and wherein the patient had an NRS≥3 prior to initiating the treatment.

In some embodiments, a reduction in an experience of flare is achieved relative to that in a patient who has not received the treatment.

In some embodiments, the patient achieves, at 16 weeks after the first daily administration, relative to that in a patient who has not received the treatment, an improvement from baseline an improvement from Baseline in Dermatology Life Quality Index (DLQI).

In some embodiments, the patient achieves, at 16 weeks after the first daily administration, relative to that in a patient who has not received the treatment, an improvement from baseline an improvement from Baseline in Hidradenitis Suppurativa Symptom Assessment (HSSA).

In some embodiments, the patient achieves, at 16 weeks after the first daily administration, relative to that in a patient who has not received the treatment, an improvement from baseline an improvement from Baseline in International Hidradenitis Suppurativa Severity Score System score.

In some embodiments, the patient achieves, at 16 weeks after the first daily administration, relative to that in a patient who has not received the treatment, an improvement from baseline an improvement from Baseline in HS-related odor, assessed based on the HSSA.

In some embodiments, the patient achieves, at 16 weeks after the first daily administration, relative to that in a patient who has not received the treatment, an improvement from baseline an improvement from Baseline in Hidradenitis Suppurativa Impact Assessment (HSIA).

In some embodiments, the patient achieves “much improved” or “very much improved” on the Total Patient Global Impression of Change (PGIC-Total) at 16 weeks after the first daily administration.

In some embodiments, the patient achieves ≥1 grade improvement in Total Patient Global Impression of Severity relative to Baseline at 16 weeks after the first daily administration.

In some embodiments, the patient achieves an improvement from Baseline in Euro-QoL 5 Dimensions 5 Levels Health State (EQ-5D-5L) at 16 weeks after the first daily administration.

In some embodiments, the patient achieves an improvement from Baseline in Work Productivity and Impairment (WPAI) at 16 weeks after the first daily administration.

In some embodiments, the treatment provides a reduction in the incidence of disease progression over 16 weeks of treatment.

In some embodiments, the treatment provides a reduction in cumulative analgesic use for HS-related skin pain relative to that in a patient who has not received the treatment at one or more of 16 weeks, 28 weeks, 36 weeks, 52 weeks, and 104 weeks.

In some embodiments, the method comprises orally administering once daily to the patient an induction dose of 30 mg of upadacitinib for 16 weeks, followed by orally administering once daily to the patient a maintenance dose of 15 mg of upadacitinib.

### BRIEF DESCRIPTION OF THE DRAWINGS

FIG. 1 is a schematic illustration of a clinical study according to an embodiment of the disclosure.

FIG. 2 is a graphical depiction of response rate over time with respect to the primary endpoint (HiSCR) for subjects treated with placebo and upadacitinib 30 mg QD.

FIG. 3 is a graphical depiction of response rate over time with respect to the secondary endpoint (Pain NRS30) for subjects treated with placebo and upadacitinib 30 mg QD.

FIG. 4 is a graphical depiction of response rate over time with respect to the secondary endpoint (NRI-C) for subjects treated with placebo and upadacitinib 30 mg QD.

FIG. 5A is a graphical depiction of the proportion of patients achieving the primary endpoint (HiSCR) at week 12 (NRI-C).

FIG. 5B is a graphical depiction of the proportion of patients achieving the secondary endpoint (Pain NRS30) at week 12 (NRI-C).

FIG. 6A is a graphical depiction of the proportion of patients achieving the primary endpoint (HiSCR) through week 40 based on non-responder imputation.

FIG. 6B is a graphical depiction of the proportion of patients achieving the primary endpoint (HiSCR) through week 40 based on observed cases.

FIG. 7A is a graphical depiction of the proportion of patients achieving the primary endpoint (HiSCR) at week 12 by TNF-α inhibitor exposure status.

FIG. 7B is a graphical depiction of the proportion of patients achieving the primary endpoint (HiSCR) at week 12 by Hurley stage (NRI-C).

### DETAILED DESCRIPTION OF THE DISCLOSURE

This written description uses examples to disclose the invention and also to enable any person skilled in the art to practice the invention, including making and using any of the disclosed compositions, and performing any of the disclosed methods or processes. The patentable scope of the invention is defined by the claims, and may include other examples that occur to those skilled in the art. Such other examples are intended to be within the scope of the claims if they have elements that do not differ from the literal language of the claims, or if they include equivalent elements.

### I. Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art.

In some embodiments, moderate to severe HS is defined herein as a total AN count of ≥5, the presence of HS lesions in at least 2 distinct anatomic areas, and a draining fistula count of ≤20.

The term “Hidradenitis Suppurativa Clinical Response 90” or “HiSCR 90” as used herein is defined as at least a 90% reduction in the total inflammatory lesion (abscess and nodule) count (AN count) relative to baseline with no increase in abscess count and no increase in draining fistula count relative to baseline.

The term “Hidradenitis Suppurativa Clinical Response 75” or “HiSCR 75” as used herein is defined as at least a 75% reduction in the total inflammatory lesion (abscess and nodule) count (AN count) relative to baseline with no increase in abscess count and no increase in draining fistula count relative to baseline.

The term “Hidradenitis Suppurativa Clinical Response 50” or “HiSCR 50” as used herein is defined as at least a 50% reduction in the total inflammatory lesion (abscess and nodule) count (AN count) relative to baseline with no increase in abscess count and no increase in draining fistula count relative to baseline.

The term “Numeric Rating Scale 30 (NRS30)” is defined as at least about 30% reduction in and at least a 1-unit reduction baseline in Numeric Rating Scale (NRS) in Patient's Global Assessment of HS-related skin pain. Numerical rating scale 30 is based on worst skin pain in a 24-hour recall period (maximal daily pain). The NRS is a segmented numeric version of the visual analog scale in which a respondent selects a whole number (0-10) that best reflects the intensity of his/her pain, with 10 being the highest.

Those “in need of treatment” include mammals, such as humans, already having hidradenitis suppurativa, including those in which the disease or disorder is to be prevented.

The term “treatment,” as used within the context of the present disclosure, is meant to include therapeutic treatment, as well as prophylactic or suppressive measures, for the treatment of hidradenitis suppurativa. For example, the term treatment may include administration of upadacitinib prior to or following the onset of hidradenitis suppurativa thereby preventing or removing signs of the disease or disorder. As another example, administration of upadacitinib after clinical manifestation of hidradenitis suppurativa to combat the symptoms and/or complications and disorders associated with hidradenitis suppurativa comprises “treatment” of the disease. Further, administration of the agent after onset and after clinical symptoms and/or complications have developed where administration affects clinical parameters of the disease or disorder and perhaps amelioration of the disease, comprises “treatment” of the hidradenitis suppurativa.

The terms “subject” and “patient”, as used herein, are used interchangeably. In one embodiment, a subject refers to an individual who may be treated therapeutically with upadacitinib.

### II. JAK1 Inhibition

Upadacitinib (ABT-494; 3 S,4R)-3-ethyl-4-(3H-imidazo[1,2-a]pyrrolo[2,3-e]pyrazin-8-yl)-N-(2,2,2-trifluoroethyl)pyrrolidine-1-carboxamide; C₁₇H₁₉F₃N₆O), or a pharmaceutically acceptable salt or solid state form thereof, is an oral Janus kinase (JAK) inhibitor that displays unique selectivity for the JAK1 receptor. Particularly, upadacitinib inhibits JAK1 with minimal inhibitory effects on JAK2 and JAK3, which could potentially minimize some of the reported safety concerns with non-selective JAK inhibition which are thought to be mediated by inhibition of JAK2 and JAK3 signaling pathways. Upadacitinib has the structure shown below:

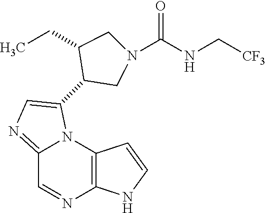

The dosage strength of upadacitinib recited in the present application is based on the weight of anhydrous freebase upadacitinib present in the active ingredient delivered to the patient. For example, a dose of “15 mg of upadacitinib” or “UPA 15 MG” refers to the 15 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “15 mg of upadacitinib” includes the administration of 15.4 mg of crystalline upadacitinib freebase hemihydrate (which includes ½ of a water conformer molecule per upadacitinib freebase molecule) which delivers 15 mg of anhydrous freebase upadacitinib to a patient.

The dosage strength of upadacitinib recited in the present application is based on the weight of anhydrous freebase upadacitinib present in the active ingredient delivered to the patient. For example, a dose of “30 mg of upadacitinib” or “UPA 30 MG” refers to the 30 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “30 mg of upadacitinib” includes the administration of 30.7 mg of crystalline upadacitinib freebase hemihydrate (which includes ½ of a water conformer molecule per upadacitinib freebase molecule) which delivers 30 mg of anhydrous freebase upadacitinib to a patient.

The dosage strength of upadacitinib recited in the present application is based on the weight of anhydrous freebase upadacitinib present in the active ingredient delivered to the patient. For example, a dose of “45 mg of upadacitinib” or “UPA 45 MG” refers to the 45 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “45 mg of upadacitinib” includes the administration of 46.1 mg of crystalline upadacitinib freebase hemihydrate (which includes ½ of a water conformer molecule per upadacitinib freebase molecule) which delivers 45 mg of anhydrous freebase upadacitinib to a patient.

Upadacitinib is approved under the brand name Rinvoq® for the treatment of patients with rheumatoid arthritis, psoriatic arthritis, atopic dermatitis, ankylosing spondylitis and ulcerative colitis.

### III. Treatment of Hidradenitis Suppurativa (HS) With Upadacitinib

The present disclosure generally provides methods for treating a human patient having hidradenitis suppurativa (HS), the methods comprising administering the selective JAK1 inhibitor upadacitinib to the patient. The disclosed methods generally comprise orally administering the upadacitinib to the patient. The upadacitinib is administered in a therapeutically effective amount. The disclosed methods generally comprise orally administering the upadacitinib to the patient daily for a period of time.

Accordingly, in one aspect is provided a method for treating a human patient having moderate to severe hidradenitis suppurativa (HS), the method comprising orally administering once daily to the patient 30 mg of upadacitinib.

The age of the patient may vary. In some embodiments, the patient is an adult patient (e.g., at least 18 years of age). In some embodiments, the patient is an adolescent patient (e.g., from about 12 to about 18 years of age). In some embodiments, the patient is at least 12 years of age.

The method comprises orally administering upadacitinib to the patient daily for a period of time. The period of time may vary. In some embodiments, the period of time is at least 12 weeks. In some embodiments, the period of time is up to 12 weeks. In some embodiments, the period of time is at least 16 weeks, such as 16 weeks, 20 weeks, 24 weeks, 28 weeks, 36 weeks, 44 weeks, 52 weeks, 64 weeks, 76 weeks, 88 weeks, 100 weeks, or 104 weeks.

In some embodiments, the method comprises orally administering upadacitinib to the patient daily for up to 16 weeks. In some embodiments, the method comprises orally administering upadacitinib to the patient daily for up to 20 weeks. In some embodiments, the method comprises orally administering upadacitinib to the patient daily for up to 24 weeks. In some embodiments, the method comprises orally administering upadacitinib to the patient daily for up to 28 weeks. In some embodiments, the method comprises orally administering upadacitinib to the patient daily for up to 36 weeks. In some embodiments, the method comprises orally administering upadacitinib to the patient daily for up to 44 weeks. In some embodiments, the method comprises orally administering upadacitinib to the patient daily for up to 52 weeks. In some embodiments, the method comprises orally administering upadacitinib to the patient daily for up to 64 weeks. In some embodiments, the method comprises orally administering upadacitinib to the patient daily for up to 76 weeks. In some embodiments, the method comprises orally administering upadacitinib to the patient daily for up to 88 weeks. In some embodiments, the method comprises orally administering upadacitinib to the patient daily for up to 100 weeks. In some embodiments, the method comprises orally administering upadacitinib to the patient daily for up to 104 weeks.

In some embodiments, the method comprises orally administering once daily to the patient an induction dose of 30 mg of upadacitinib for 16 weeks, followed by orally administering once daily to the patient a maintenance dose of 15 mg of upadacitinib.

In some embodiments, the patient has moderate HS. In some embodiments, the patient has severe HS. In some embodiments, the severity of HS prior to initiating the treatment is determined according to the Hurley staging system. Hurley staging is based on assigning the subject having HS one of three different “Stages” depending on the disease level. More specifically, Stage I refers to abscess formation, single or multiple, without sinus tracts and cicatrisation; Stage II refers to recurrent abscesses with tract formation and cicatrisation, as well as single or multiple, widely separated lesions; and Stage III, which refers to diffuse or near-diffuse involvement, or multiple interconnected tracts and abscesses across the entire area. Hurley Stage III is the most severe form, reflecting diffuse or near-diffuse involvement of affected areas. See, for example, Poli et al., Clinical Presentation; In Hidradenitis Suppurativa. Jemec et al., editors, Springer, New York, 2006, pp 11-24, incorporated herein by reference.

In some embodiments, the patient has HS lesions in at least two distinct anatomic areas prior to treatment. In one embodiment, the subject having HS has HS lesions that are present in at least two distinct anatomic areas (e.g., left and right axilla; or left axilla and left inguinal-crural fold), one of which is at least Hurley Stage II. In another embodiment, the subject being treated has at least one lesion that is at least a Hurley Stage II. In some embodiments, the patient has a total AN count equal to or greater than 5 prior to initiating the treatment. In some embodiments, the patient has a draining fistula count of less than or equal to 20 prior to initiating the treatment.

In some embodiments, the patient has failed to respond to or was intolerant of anti-TNF therapy. In some embodiments, the patient has been unresponsive to or intolerant to oral antibiotics for treatment of their hidradenitis suppurativa. In one embodiment, such patients have had a diagnosis of moderate to severe hidradenitis suppurativa for at least 1 month, such as at least 6 months prior to Baseline, and the HS involves at least two distinct anatomic areas (e.g., left and right axilla; or left axilla and left inguinal-crural fold).

The methods disclosed herein generally provides an improvement in one or more aspects of HS based on indices used to measure the disease state. Treatment efficacy of HS using upadacitinib may be determined using measures known in the art, including those measures described herein. In some embodiments, after receiving the treatment for a period of time, the patient achieves a clinical response. The term “clinical response” as used herein refers to an indicator of therapeutic effectiveness of upadacitinib, such as a particular HiSCR, or an improvement in one or more symptom assessments.

In some embodiments, the patient achieves a Hidradenitis Suppurativa Clinical Response 50 (HiSCR50) at 12 weeks after the first daily administration. In some embodiments, a Hidradenitis Suppurativa Clinical Response 50 (HiSCR 50) is achieved at Week 16. In some embodiments, a Hidradenitis Suppurativa Clinical Response 75 (HiSCR 75) is achieved at Week 16. In some embodiments, a Hidradenitis Suppurativa Clinical Response 90 (HiSCR 90) is achieved at Week 16.

In some embodiments, a Numerical Rating Scale 30 (NRS30) is achieved in a Patient's Global Assessment of HS-related skin pain at Week 2, wherein the patient had an NRS≥3 prior to initiating the treatment.

In some embodiments, the number of inflammatory lesions (AN count) is decreased in the patient relative to an AN count prior to initiating treatment. In some embodiments, the AN count is decreased by at least 50% at 12 weeks after the first daily administration, relative to an AN count prior to initiating treatment.

In some embodiments, the methods disclosed herein result in a reduction in an experience of flare relative to that in a patient who has not received the treatment, wherein flare is defined as an increase in AN count of at least 25% with a minimum increase of 2 relative to Baseline.

In some embodiments, the methods disclosed herein result in a reduction in the incidence of HS progression, where HS progression is defined as at least 1 of the following: lesion spread, scar spread, and progression of Hurley Stage at any anatomic region.

In some embodiments, the patient achieves, at 12 weeks after the first daily administration, relative to that in a patient who has not received the treatment, an improvement from baseline in Dermatology Life Quality Index (DLQI). In some embodiments, the patient achieves, at 16 weeks after the first daily administration, relative to that in a patient who has not received the treatment, an improvement from baseline an improvement from Baseline in DLQI. The DLQI consists of 10 questions concerning patients' perception of the impact of skin diseases on different aspects of their health-related quality of life over the last week.

In some embodiments, the patient achieves, at 12 weeks after the first daily administration, relative to that in a patient who has not received the treatment, an improvement from baseline in Hidradenitis Suppurativa Symptom Assessment (HSSA) is achieved, relative to that in a patient who has not received the treatment. In some embodiments, the patient achieves, at 16 weeks after the first daily administration, relative to that in a patient who has not received the treatment, an improvement from baseline an improvement from Baseline in HSSA. The HSSA is a patient-reported outcome (PRO) questionnaire developed to measure signs, symptoms and impacts of HS in treatment efficacy studies.

In some embodiments, the patient achieves, at 12 weeks after the first daily administration, relative to that in a patient who has not received the treatment, an improvement from baseline in HS-related swelling, assessed based on the HSSA is achieved, relative to that in a patient who has not received the treatment. In some embodiments, the patient achieves, at 12 weeks after the first daily administration, relative to that in a patient who has not received the treatment, an improvement from baseline in HS-related odor, assessed based on the HSSA is achieved, relative to that in a patient who has not received the treatment.

In some embodiments, the patient achieves, at 12 weeks after the first daily administration, relative to that in a patient who has not received the treatment, an improvement from baseline in HS-related worst drainage, assessed based on the HSSA, is achieved, relative to that in a patient who has not received the treatment.

In some embodiments, a draining fistula count is decreased in the patient relative to a draining fistula count prior to initiating treatment.

In some embodiments, the patient achieves, at 16 weeks after the first daily administration, relative to that in a patient who has not received the treatment, an improvement from baseline an improvement from Baseline in International Hidradenitis Suppurativa Severity Score System score.

In some embodiments, the patient achieves, at 16 weeks after the first daily administration, relative to that in a patient who has not received the treatment, an improvement from baseline an improvement from Baseline in HS-related odor, assessed based on the HSSA.

In some embodiments, the patient achieves, at 16 weeks after the first daily administration, relative to that in a patient who has not received the treatment, an improvement from baseline an improvement from Baseline in Hidradenitis Suppurativa Impact Assessment (HSIA).

In some embodiments, the patient achieves “much improved” or “very much improved” on the Total Patient Global Impression of Change (PGIC-Total) at 16 weeks after the first daily administration.

In some embodiments, the patient achieves ≥1 grade improvement in Total Patient Global Impression of Severity relative to Baseline at 16 weeks after the first daily administration.

In some embodiments, the patient achieves an improvement from Baseline in Euro-QoL 5 Dimensions 5 Levels Health State (EQ-5D-5L) at 16 weeks after the first daily administration.

In some embodiments, the patient achieves an improvement from Baseline in Work Productivity and Impairment (WPAI) at 16 weeks after the first daily administration.

In some embodiments, the treatment provides a reduction in the incidence of disease progression over 16 weeks of treatment.

In some embodiments, the treatment provides a reduction in cumulative analgesic use for HS-related skin pain relative to that in a patient who has not received the treatment at one or more of 16 weeks, 28 weeks, 36 weeks, 52 weeks, and 104 weeks.

### IV. Pharmaceutical Compositions and Routes of Administration

Upadacitinib can be administered to a human patient by itself or in pharmaceutical composition where it is mixed with biologically suitable carriers or excipient(s) at doses to treat or ameliorate a disease or condition as described herein. Mixtures of these compounds can also be administered to the patient as a simple mixture or in suitable formulated pharmaceutical compositions.

The pharmaceutical compositions of the present disclosure may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

### EXEMPLIFICATION

Examples 1 and 2 demonstrate that the JAK1 inhibitor, upadacitinib, is efficacious and safe for treating hidradenitis suppurativa (HS) in human patients, especially human patients with moderate to severe chronic HS.

### Example 1. Safety and Efficacy of Upadacitinib in Subjects With Moderate to Severe Chronic Hidradenitis Suppurativa (HS)

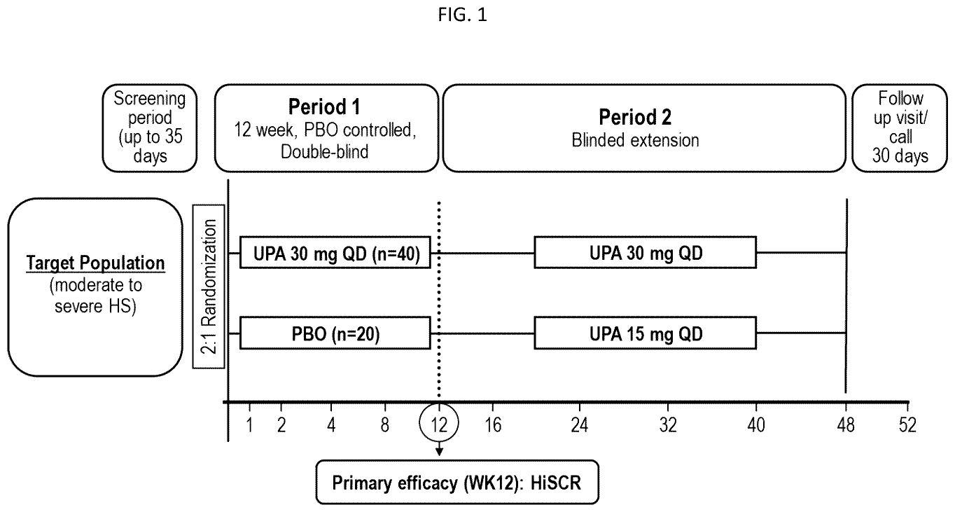

This study was a Phase 2, multicenter, randomized, double-blind, parallel-group, placebo-controlled study to evaluate the efficacy and safety of upadacitinib in adult human subjects with moderate to severe Hidradenitis Suppurativa (HS). FIG. 1 shows the study design for this clinical trial.

### Study Design

The duration of the study was 57 weeks, including an approximately 35-day screening period followed by a 48-week double-blinded treatment period and a 30-day follow-up visit after the last dose of study drug. Subjects were randomized in a 2:1 ratio to 1 of the 2 arms as shown below:
Upadacitinib 30 mg once daily (QD) (N=40): Daily oral doses of upadacitinib 30 mg from the Baseline visit through Period 1 and Period 2.
Placebo (N=20): Placebo for upadacitinib from the Baseline visit up to the Week 12 visit (Period 1). At Week 12, subjects were switched to blinded upadacitinib 15 mg QD through Period 2.

### Objectives and Efficacy Endpoints

The primary objective of this study was to assess the efficacy and safety of upadacitinib 30 mg QD in adult subjects with moderate to severe HS. The primary efficacy objective was assessed based on the achievement of HiSCR after 12 weeks treatment.

### Primary Endpoint

The primary endpoint was Hidradenitis Suppurativa Clinical Response (HiSCR) at Week 12, where HiSCR is defined as at least 50% reduction in the total abscess and inflammatory nodule (AN) count with no increase in abscess count and no increase in draining fistula count relative to Baseline.

### Secondary Endpoint

The secondary endpoint was Pain Numerical Rating Scale (NRS30) at Week 12: the achievement of at least 30% reduction and at least 1 unit reduction from Baseline in NRS30 in Patient's Global Assessment of Skin Pain (PGA Skin Pain) at Week 12, among subjects with Baseline NRS≥3.

### Additional Endpoints

The following additional efficacy endpoints were evaluated:
1. Experience of flare, defined as an increase in AN count of at least 25% with a minimum increase of 2 relative to Baseline
2. Change from Baseline in Dermatology Life Quality Index (DLQI)
3. Change from baseline in Hidradenitis Suppurativa Symptom Assessment (HSSA)
4. Change from baseline in HS-related swelling, assessed based on the HSSA
5. Change from baseline in HS-related odor, assessed based on the HSSA
6. Change from baseline in HS-related worst drainage, assessed based on the HSSA

For the primary and secondary endpoints, comparisons of upadacitinib vs. placebo were made with 3 different analysis strategies, as summarized in Table 1 below:

TABLE 1
Endpoint Analysis Strategies

| | | | | |
|---|---|---|---|---|
| | | Historical Data | Analysis | Significance |
| Analysis | Comparator | Used | Method | Criterion |

Primary Endpoint: HiSCR at Week 12

| | | | | |
|---|---|---|---|---|
| Primary | Historical | Placebo subjects | One sample | 1-sided |
| | placebo rate | from Humira | Chi-square test | p-value ≤ |
| | (25%) | Studiesa | | 0.05 |
| | | (N = 259) | | |
| Sensitivity 1 | Synthetic PBO | Subject-level | CMH test | 1-sided |
| | plus in-trial PBO | data from | | p-value ≤ |
| | | Humira and | | 0.05 |
| | | Risankizumab | | |
| | | studies using | | |
| | | propensity score | | |
| | | matching (N = 68) | | |
| Sensitivity 2 | In-trial PBO | None | CMH test | 1-sided |
| | | | | p-value ≤ |
| | | | | 0.05 |

Secondary endpoint: Pain NRS30 at Week 12 Among
Subjects with Baseline NRS ≥ 3

| | | | | |
|---|---|---|---|---|
| Primary | Historical | Placebo subjects | One sample | 1-sided |
| | placebo rate | from Humira | Chi-square test | p-value ≤ |
| | (22.5%) | Studies^{a} (N = 259) | | 0.05 |
| Sensitivity 1 | Synthetic PBO | Subject-level | CMH test | 1-sided |
| | plus in-trial PBO | data from | | p-value ≤ |
| | | Humira and | | 0.05 |
| | | Risankizumab | | |
| | | studies using | | |
| | | propensity score | | |
| | | matching (N = 68) | | |
| Sensitivity 2 | In-trial PBO | None | CMH test | 1-sided |
| | | | | p-value ≤ |
| | | | | 0.05 |

^{a}Historical placebo rate of HISCR (25%) and NRS30 (22.5%): assumed from placebo subjects who fulfilled the same eligibility criteria from prior adalimumab HS studies (Pioneer I and Pioneer II).

For the additional endpoints, comparisons were made for upadacitinib vs. historical reference value of placebo, and upadacitinib vs. in-trial placebo subjects.

### Primary Approach to Handle Missing Data

For categorical variables, the primary approach to handle missing data was Non-Responder Imputation [NRI] incorporating Multiple Imputation [MI]. For continuous variables, missing data was handled by Mixed-effect Model Repeat Measurement (MMRM). Missing data for long-term efficacy in Period 2 were handled based on the observed case (OC).

### Study Population and Number of Subjects Enrolled

A total of 68 subjects were randomized (21 in PBO and 47 in UPA 30 mg). A total of 60 (88.2%) subjects completed study drug in Period 1 (Table 2). Demographics and baseline characteristics were generally balanced between treatment groups (Table 3). Subject disposition in the ongoing Period 2 is presented in Table 4.

TABLE 2
Subject Disposition in Period 1

| | PBO | | UPA 30 MG | | Total | |
|---|---|---|---|---|---|---|
| | (N = 21) | | (N = 47) | | (N = 68) | |
| Subject Disposition | n (%) | | n (%) | | n (%) | |
| Randomized | 21 | (100) | 47 | (100) | 68 | (100) |
| Randomized but not Treated | 0 | | 0 | | 0 | |
| Treated | 21 | (100) | 47 | (100) | 68 | (100) |
| Completed the study drug | 19 | (90.5) | 41 | (87.2) | 60 | (88.2) |
| Discontinued Study Drug during the | 2 | (9.5) | 6 | (12.8) | 8 | (11.8) |
| Period 1(All Reasons) | | | | | | |
| Adverse Event | 1 | (4.8) | 1 | (2.1) | 2 | (2.9) |
| Withdrew Consent | 1 | (4.8) | 2 | (4.3) | 3 | (4.4) |
| Lack of efficacy | 0 | | 1 | (2.1) | 1 | (1.5) |
| Lost to follow-up | 0 | | 2 | (4.3) | 2 | (2.9) |
| COVID-19 infection or related logistic | 0 | | 0 | | 0 | |
| restrictions | | | | | | |
| Other | 1 | (4.8) | 1 | (2.1) | 2 | (2.9) |

TABLE 3
Key Demographics and Disease Characteristics

| | PBO | | UPA 30 MG | |
|---|---|---|---|---|
| Parameter | (N = 21) | | (N = 47) | |
| Age (years), mean (SD) | 36.6 | (12.5) | 36.7 | (11.7) |
| Female, n (%) | 16 | (76.2) | 37 | (78.7) |
| Race (White), n (%) | 11 | (52.4) | 30 | (63.8) |
| Body Mass Index (kg/M²), mean | 34.2 | (6.5) | 35.8 | (9.4) |
| (SD) | | | | |
| Disease duration (years), mean (SD) | 9.8 | (5.0) | 11.5 | (9.4) |
| Prior exposure to anti-TNF biologics, | 6 | (28.6) | 12 | (25.5) |
| n(%) | | | | |
| Hurley Stage, n (%) | | | | |
| I | 1 | (4.8) | 0 | |
| II | 11 | (52.4) | 24 | (51.1) |
| III | 9 | (42.9) | 23 | (48.9) |
| Total abscess and inflammatory | 19.8 | (12.9) | 23.2 | (24.6) |
| nodule count (AN count), mean (SD) | | | | |
| Abscess count, mean (SD) | 4.0 | (3.3) | 4.8 | (6.4) |
| Inflammatory nodule count, mean | 15.7 | (12.8) | 18.4 | (21.0) |
| (SD) | | | | |
| Draining fistulas, mean (SD) | 4.3 | (6.17) | 3.4 | (4.74) |
| PGA Skin Pain in NRS, mean (SD) | 4.9 | (3.3) | 5.0 | (2.7) |
| hs-CRP (mg/L), Median (range) | 5.3 | (0.2-117) | 7.2 | (0.5-108.7) |
| DLQI Total Score, Mean (SD | 5.000 | (8.6987) | 13.311 | (7.5553) |
| HSSA Total Score, Mean (SD) | 4.455 | (2.9278) | 4.493 | (2.4502 |
| HSSA Swelling Score, Mean (SD) | 5.097 | (3.2656) | 4.819 | (2.7208 |
| HSSA Odor Score, Mean (SD) | 3.573 | (3.1562) | 3.907 | (3.2520) |
| HSSA Worst Drainage Score, Mean | 4.025 | (3.1971) | 4.055 | (2.8944) |
| (SD) | | | | |

TABLE 4
Subject Disposition in Period 2

| | Placebo/UPA 15 mg | | UPA 30 mg | | Overall |
|---|---|---|---|---|---|
| | (N = 19) | | (N = 47) | | (N = 66) |
| Subject Disposition | n (%) | | n (%) | | n (%) |
| Entered Period 2 and | 19 | (100) | 41 | (87.2) | 60 (90.9) |
| received study drug | | | | | |
| Completed Study | 0 | | 0 | | 0 |
| Drug in Period 2 | | | | | |
| Discontinued Study | 1 | (5.3) | 2 | (4.3) | 3 (4.5) |
| Drug during Period 2 | | | | | |
| (Primary Reasons) | | | | | |
| Adverse event | 0 | | 0 | | 0 |
| Withdrew consent | 0 | | 2 | (4.3) | 2 (3.0) |
| Lost to follow-up | 0 | | 0 | | 0 |
| Lack of efficacy | 0 | | 1 | (2.1) | 1 (1.5) |
| COVID-19 infection | 0 | | 0 | | 0 |
| COVID-19 logistical | 0 | | 0 | | 0 |
| restrictions | | | | | |
| Other | 1 | (5.3) | | | 1 (1.5) |

Note:
Subjects who were randomized to placebo in Period 1 and did not continue into Period 2 are excluded from this summary.

### Efficacy

### Primary and Secondary Endpoints

The study met the primary endpoint and demonstrated superiority of upadacitinib 30 mg vs. the historical placebo rate (1-sided p-value=0.018). Results from the primary and secondary endpoints are summarized in Table 5. The results from selected additional endpoints are provided in Table 6.

### HiSCR at Week 12 (NRI-C):

Treatment with upadacitinib 30 mg demonstrated a statistically significantly greater percentage of subjects achieving a HiSCR response at Week 12 (38.3%) compared with historical placebo rate of 25%.
Sensitivity analysis of upadacitinib vs. synthetic placebo combined with in-trial placebo subjects showed similar results favoring UPA 30 mg (38.3% vs. 29.2% in placebo, 1-sided p-value=0.142).

Sensitivity analysis of upadacitinib vs. in-trial placebo subjects only showed similar results favoring UPA 30 mg (38.3% vs. 23.8% in placebo, 1-sided p-value=0.087).

TABLE 5
Primary and Secondary Endpoints (NRI-C)

| | | | Sensitivity Analysis 1: | | | |
|---|---|---|---|---|---|---|
| | | | UPA vs. Synthetic PBO | | | |
| | | | plus In-Trial PBO | | Sensitivity Analysis 2: | |
| | Primary Analysis: UPA | | PBO | | UPA vs. In-Trial PBO | |
| | vs. Historical PBO Rate | | (Synthetic | | PBO | |
| | Historical | UPA 30 mg | plus In-trial) | UPA 30 mg | (In-trial) | UPA 30 mg |
| Endpoint | PBO Rate | (N = 47) | (N = 89) | (N = 47) | (N = 21) | (N = 47) |

Primary Endpoint: HiSCR at Week 12

| | | | | | | |
|---|---|---|---|---|---|---|
| n (%) | 25% | 18 (38.3%) | 26 (29.2%) | 18 (38.3%) | 5 (23.8%) | 18 (38.3%) |
| UPA - | | Diff = 13.3% | | Diff = 9.2% | | Diff = 14.7% |
| PBO | | p = 0.018* | | p = 0.142 | | p = 0.087 |
| 1-sided p- | | | | | | |
| value | | | | | | |

Secondary Endpoint: Pain NRS30 at Week 12 Among Subjects with Baseline NRS ≥ 3

| | | | | | | |
|---|---|---|---|---|---|---|
| Nn (%) | 22.5% | N = 33 | N = 80 | N = 33 | N = 12 | N = 33 |
| UPA - | | 12 (36.4%) | 25 (31.3%) | 12 (36.4%) | 4 (33.3%) | 12 (36.4%) |
| PBO | | Diff = 13.9% | | Diff = 4.5% | | Diff = 2.2% |
| 1-sided p- | | p = 0.028* | | p = 0.323 | | p = 0.421 |
| value | | | | | | |

*1-sided p-value ≤ 0.05.

TABLE 6
Selected Additional Endpoints

| | UPA vs. Historical PBO Rate/ | | | | |
|---|---|---|---|---|---|
| | Historical Reference Value of PBO | | | | |
| | Historical | | | | |
| | PBO Rate/ | | UPA vs. In-Trial PBO | | |
| | Historical | | PBO | | |
| | Reference | UPA 30 mg | (In-Trial) | | UPA 30 mg |
| Endpoint | Value of PBO | (N = 47) | (N = 21) | | (N = 47) |
| Flare during | 34% | 6 (12.8%) | 4 | (19%) | 6 (12.8%) |
| Period 1 | | Diff = −21.2 | | | Diff = −6.5 |
| n (%) | | p = 0.001* | | | p = 0.231 |
| UPA - PBO | | | | | |
| 1-sided p-value | | | | | |
| Change in DLQI | −2.5 | −2.4 (1.05) | −1.0 | (1.63) | 2.4 (1.03) |
| at Week 12 | | Diff = 0.1 | | | Diff = −1.4 |
| LS Mean (SE) | | p = 0.548 | | | p = 0.222 |
| UPA - PBO | | | | | |
| 1-sided p-value | | | | | |
| Change in HSSA | −0.72 | 0.969 (0.28) | −0.272 | (0.37) | −1.034 (0.27) |
| total symptom | | Diff = −0.249 | | | Diff = −0.762 |
| at Week 12 | | p = 0.188 | | | p = 0.046* |
| LS Mean (SE) | | | | | |
| UPA - PBO | | | | | |
| 1-sided p-value | | | | | |
| Change in HSSA | −0.8 | 0.931 (0.37) | −0.127 | (0.48) | −0.922 (0.34) |
| swelling at | | Diff = −0.131 | | | Diff = −0.795 |
| Week 12 | | p = 0.364 | | | p = 0.086 |
| LS Mean (SE) | | | | | |
| UPA - PBO | | | | | |
| 1-sided p-value | | | | | |
| Change in HSSA | −0.72 | 1.199 (0.32) | 0.075 | (0.51) | −1.171 (0.36) |
| odor at | | Diff = −0.479 | | | Diff = −1.246 |
| Week 12 | | p = 0.072 | | | p = 0.024* |
| LS Mean (SE) | | | | | |
| UPA - PBO | | | | | |
| 1-sided p-value | | | | | |
| Change in HSSA | −0.89 | −1.131 (0.31) | 0.564 | (0.42) | −1.259 (0.30) |

| | | |
|---|---|---|
| worst drainage | Diff = −0.241 | Diff = −0.695 |
| at Week 12 | p = 0.219 | p = 0.086 |
| LS Mean (SE) | | |
| UPA - PBO | | |
| 1-sided p-value | | |

*p-value ≤ 0.05.

### Efficacy Over Time

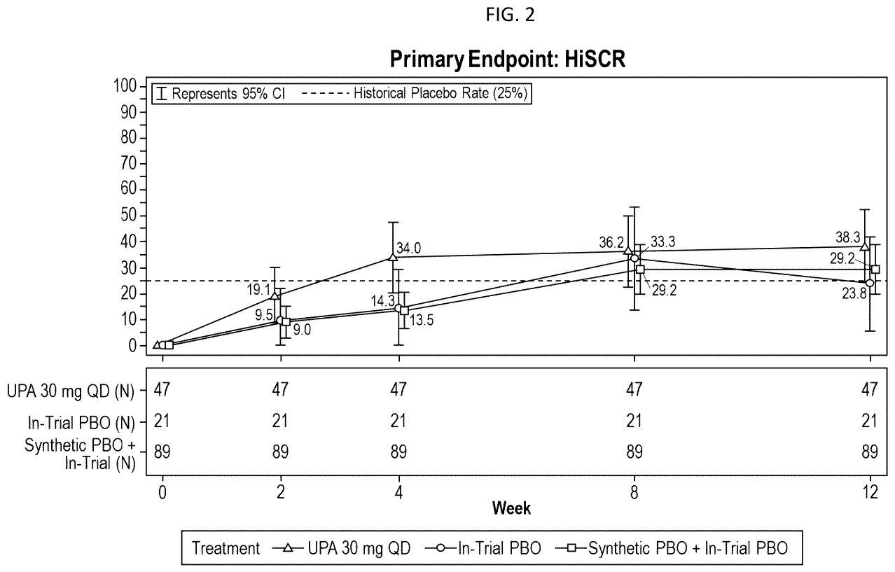

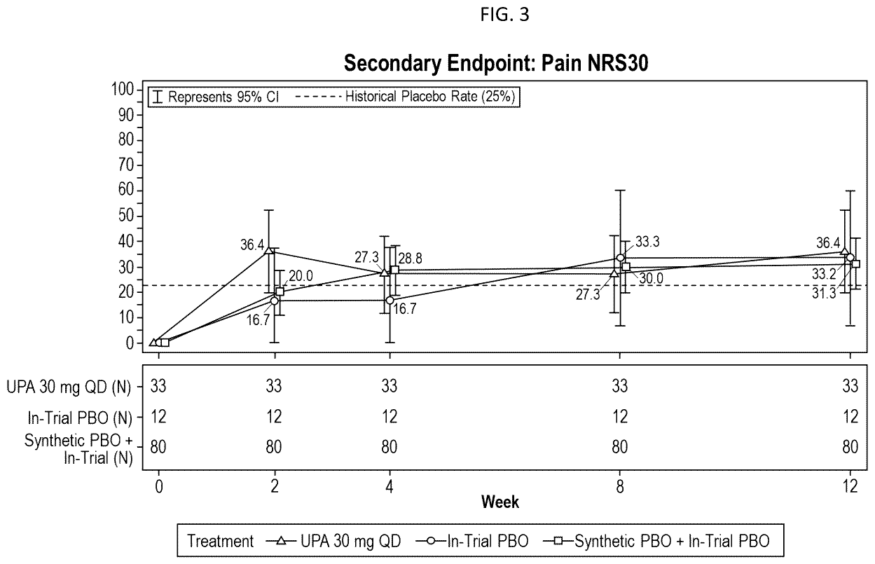

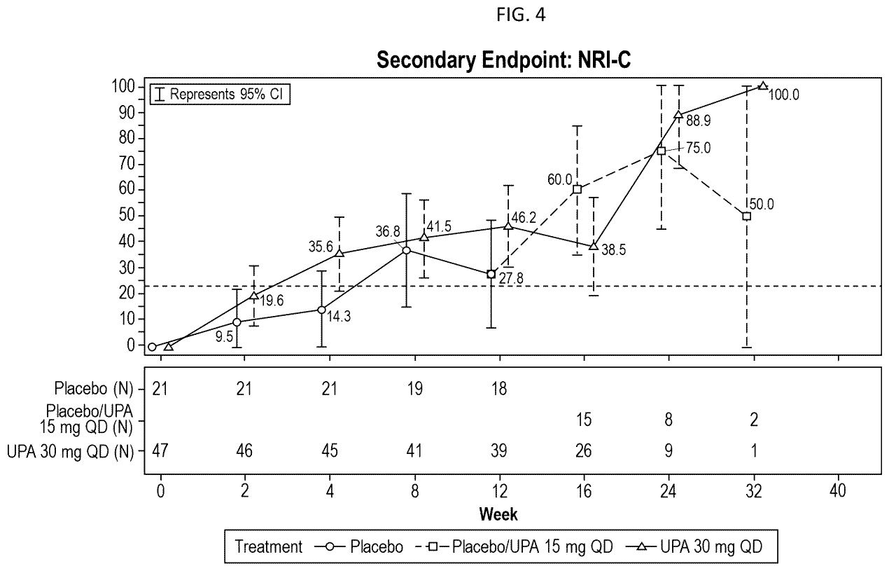

Response rates and the 95% confidence intervals (CIs) for HiSCR and Pain NRS30 at each visit in Period 1 using NRI-C are presented in FIG. 2 and FIG. 3 for the following 3 groups: UPA 30 mg; Synthetic placebo combined with in-trial placebo subjects; In-trial placebo subjects only. Particularly, FIG. 2 shows the response rates and 95% CIs for HiSCR by visit in Period 1 (NRI-C), and FIG. 3 shows the response rates and 95% CIs for Pain NRS30 by Visit in Period 1 (NRI-C). Response rates and 95% CIs for HiSCR at each visit up to the cutoff date based on OC are presented in FIG. 4 for upadacitinib 30 mg and in-trial placebo in Period 1 followed by upadacitinib 15 mg in Period 2 up to the cutoff date. Table 7 is a summary of HiSCR at Week 12 overall and by stratum.

TABLE 7
Summary of HiSCR at Week 12 overall and by Stratum

| | UPA vs. Synthetic PBO + | | | |
|---|---|---|---|---|
| | In-Trial PBO | | | |
| | PBO | | UPA vs. In-Trial PBO | |
| HiSCR at Week | (Synthetic + | | PBO | |
| 12, % (N) | In-Trial) | UPA 30 mg | (In-Trial) | UPA 30 mg |

NRI-C

| | | | | | | |
|---|---|---|---|---|---|---|
| Overall | 29.2% | (N = 89) | 38.3% (N = 47) | 23.8% | (N = 21) | 38.3% (N = 47) |
| By Stratum: | | | | | | |
| TNF-IR | 25% | (N = 12) | 41.7% (N = 12) | 16.7% | (N = 6) | 41.7% (N = 12) |
| TNF-Naïve | 29.9% | (N = 77) | 37.1% (N = 35) | 26.7% | (N = 15) | 37.1% (N = 35) |
| Hurley Stage | 27.1% | (N = 48) | 37.5% (N = 24) | 8.3% | (N = 12) | 37.5% (N = 24) |
| (<III) | | | | | | |
| Hurley Stage | 31.7% | (N = 41) | 39.1% (N = 23) | 44.4% | (N = 9) | 39.1% (N = 23) |
| (III) | | | | | | |

MI

| | | | | | | |
|---|---|---|---|---|---|---|
| Overall | 30% | (N = 89) | 39.6% (N = 47) | 23.8% | (N = 21) | 38.9% (N = 47) |
| By Stratum: | | | | | | |
| TNF-IR | 25% | (N = 12) | 41.7% (N = 12) | 16.7% | (N = 6) | 41.7% (N = 12) |
| TNF-Naïve | 30.7% | (N = 77) | 39.0% (N = 35) | 26.7% | (N = 15) | 37.9% (N = 35) |
| Hurley Stage | 28.5% | (N = 48) | 39.0% (N = 24) | 8.3% | (N = 12) | 38.1% (N = 24) |
| (<III) | | | | | | |
| Hurley Stage | 31.7% | (N = 41) | 40.3% (N = 23) | 44.4% | (N = 9) | 39.7% (N = 23) |

(III)

### Safety

Treatment-emergent adverse events (TEAEs), adverse events of interests (AESIs), and potentially clinically important (PCI) laboratory changes were monitored in Period 1. The most frequently reported TEAE (≥5%) included headache, dizziness, and urinary tract infection in the upadacitinib 30 mg QD group and myalgia, cellulitis, and dermatitis in the placebo group.

Those skilled in the art will recognize or be able to ascertain using no more than routine experimentation, many equivalents to the specific embodiments of the invention described herein. Such equivalents are intended to be encompassed by the following claims. The contents of all references, patents and published patent applications cited throughout this application are incorporated herein by reference.

### Example 2. Safety and Efficacy of Upadacitinib in Subjects with Moderate to Severe Chronic Hidradenitis Suppurativa (HS)—Post-Hoc Analysis

A post-hoc efficacy assessment through week 40 was performed in the study of Example 1. The analysis included the proportion of patients achieving ≥75% and ≥90% reduction from baseline in AN count with no increase in abscess or draining fistula count (HiSCR 75 and HiSCR 90, respectively) and the proportion of patients achieving ≥55% reduction in International HS Severity Score System endpoint (IHS4-55), which dynamically assesses inflammatory nodules, abscesses, and draining tunnels.

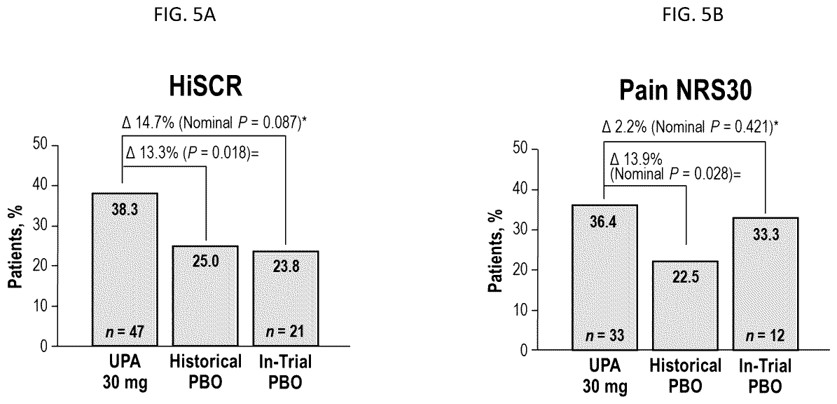

As described above, the Phase 2 study met the primary endpoint, showing that a statistically significantly greater proportion of patients with moderate-to-severe HS treated with upadacitinib 30 mg achieved HiSCR at week 12 compared with a prespecified historical placebo rate in adults by the primary analysis. Specifically, a statistically significantly greater proportion of patients receiving upadacitinib 30 mg achieved HiSCR at week 12 (primary endpoint) compared with the prespecified single historical placebo rate (upadacitinib 30 mg, 38.3%; historical placebo, 25.0%; difference=13.3% [95% CI, −0.6 to 27.2]; 1-sided P=0.018) (FIG. 5A). A higher proportion of UPA 30-mg patients achieved HiSCR 75 (21.3%, nominal P<0.001) and HiSCR 90 (8.5%, nominal P=0.015) vs placebo at week 12; and no patients in the placebo group achieved HiSCR 75 or HiSCR 90.

When the effect of upadacitinib on HiSCR was compared with in-trial placebo, a similar trend was observed, with a numerically greater proportion of patients receiving upadacitinib 30 mg achieving HiSCR (adjusted difference=14.7%; nominal P=0.087). Similar results were also observed in a supplemental analysis when the effect of upadacitinib was compared with the effect of the combined synthetic plus in-trial placebo (Table 8).

With respect to NRS30, a higher proportion of patients receiving upadacitinib 30 mg achieved this endpoint compared with the prespecified historical placebo rate. Specifically, among patients with baseline NRS≥3, a numerically greater proportion of patients receiving upadacitinib 30 mg achieved NRS30 at week 12 compared with the prespecified single historical placebo rate (secondary endpoint; upadacitinib 30 mg, 36.4% vs. historical placebo, 22.5%; difference=13.9%; nominal P=0.028) (FIG. 5B). A numerically greater proportion of patients receiving upadacitinib 30 mg achieved NRS30 at week 12 compared with patients receiving in-trial placebo (upadacitinib 30 mg 36.4% vs. in-trial placebo, 33.3%; adjusted difference=2.2%, nominal P=0.421) or compared with the combined synthetic plus in-trial placebo (Table 8).

TABLE 8
Proportion of Patients Achieving HiSCR and NRS30 at Week 12 With
Upadacitinib Compared with Synthetic Plus In-Trial Placebo

| | Upadacitinib | Synthetic Plus In- |
|---|---|---|
| Endpoint | 30 mg | Trial Placebo |
| Primary: HiSCR at week 12, n | 47 | 89 |
| Patients, n (%) | 18 (38.3) | 26 (29.2) |
| Upadacitinib versus synthetic | | |
| plus in-trial placebo | | |
| Adjusted difference (95% CI) | 9.2% (−7.6, 25.9) | |
| P value* | 0.142 | |
| Secondary: NRS30 at week 12, † n | 33 | 80 |
| Patients, n (%) | 12 (36.4) | 25 (31.3) |
| Upadacitinib versus synthetic | | |
| plus in-trial placebo | | |

| | |
|---|---|
| Adjusted difference (95% CI) | 4.5% (−14.6, 23.5) |
| Nominal P value* | 0.323 |

*1-sided P ≤ 0.05 using Cochran-Mantel-Haenszel test adjusted for strata (baseline Hurley stage [<III, III]) and prior tumor necrosis factor inhibitor use (yes or no).
† Among patients with baseline NRS ≥ 3.

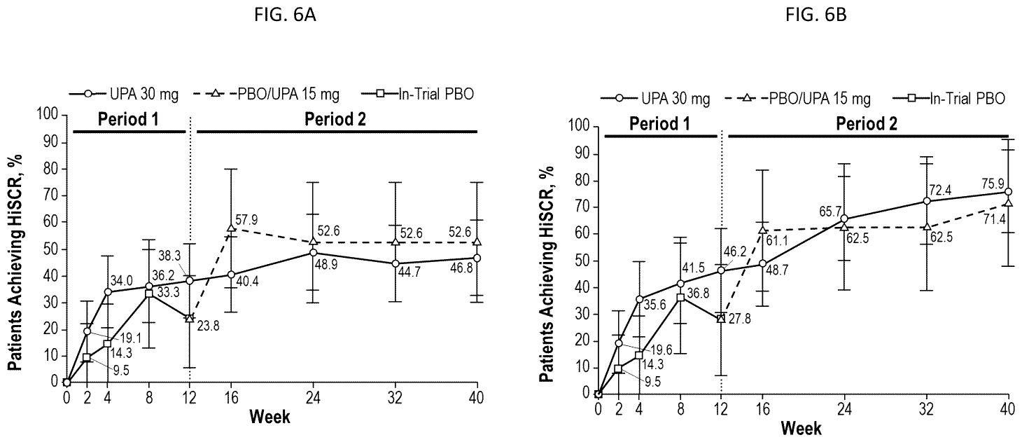

The achievement of HiSCR with upadacitinib 30 mg at week 12 continued to improve or was maintained through week 40 (FIGS. 6A and 6B and Table 9). Of the 21 patients randomized to placebo, 19 (90.5%) switched to receiving upadacitinib 15 mg; the proportion of these patients achieving HiSCR increased within the first 4 weeks of the blinded extension period, and this proportion of patients was maintained through week 40, demonstrating a trend of improvement from baseline similar to that observed in patients receiving upadacitinib 30 mg. HiSCR achievement with upadacitinib 30 mg through week 12 was independent of baseline Hurley stage or prior TNF-α inhibitor exposure and was maintained through week 40. At week 40 (OC), HiSCR, HiSCR 75, and HiSCR 90 was achieved by 75.9%, and 31.0% of patients receiving UPA 30 mg (n=29), respectively, and by 71.4%, 50.0%, and 28.6% of patients who switched to UPA 15 mg (n=14), respectively (Table 9). Given the lack of achievement by placebo-treated patients, these higher HiSCR efficacy levels may more clearly differentiate responders. The response to upadacitinib was durable with the proportions of patients achieving HiSCR 75 and HiSCR 90 increasing through week 40.

Analyses using IHS4-55 were consistent with those reported for HiSCR. Specifically, at week 40, IHS4-55, which weights draining tunnels, was achieved by 72.4% of patients receiving upadacitinib 30 mg and 85.7% of patients switching to upadacitinib 15 mg (Table 9). Among patients in the UPA 30-mg group, 40.4% achieved IHS4-55 compared with 19.0% of patients in the placebo group at week 12 (nominal P=0.020 vs placebo). The proportions of patients receiving upadacitinib 30 mg who achieved these benchmarks at week 12 were slightly higher for the OC analysis (Table 9).

TABLE 9
Achievement of Higher Clinical Efficacy with Upadacitinib Among
Patients with Moderate-to-Severe Hidradenitis Suppurativa

| | Patients, n (%) | | | | | | | |
|---|---|---|---|---|---|---|---|---|
| | NRI-C^{a} | | | | Observed Case | | | |
| | | | Week 40 | | | | Week 40 | |
| | | | UPA | | | | UPA | |
| | Week 12 | | 30 mg/ | PBO/ | Week 12 | | 30 mg/ | PBO/ |
| | UPA | | UPA | UPA | UPA | | UPA | UPA |
| | 30 mg | PBO | 30 mg | 15 mg | 30 mg | PBO | 30 mg | 15 mg |
| Parameter | (n = 47) | (n = 21) | (n = 47) | (n = 19) | (n = 39) | (n = 18) | (n = 29) | (n = 14) |
| HiSCR | 18 (38.3)* | 5 (23.8) | 22 (46.8) | 10 (52.6) | 18 (46.2) | 5 (27.8) | 22 (75.9) | 10 (71.4) |
| HiSCR 75 | 10 (21.3)^{†} | 0 | 18 (38.3) | 7 (36.8) | 10 (25.6) | 0 | 18 (62.1) | 7 (50.0) |
| HiSCR 90 | 4 (8.5)^{‡} | 0 | 9 (19.1) | 4 (21.1) | 4 (10.3) | 0 | 9 (31.0) | 4 (28.6) |
| IHS4-55 | 19 (40.4)^{§} | 4 (19.0) | 21 (44.7) | 12 (63.2) | 19 (48.7) | 4 (22.2) | 21 (72.4) | 12 (85.7) |

HiSCR, Hidradenitis Suppurativa Clinical Response defined as ≥50% reduction from baseline in abscess and inflammatory nodule (AN) count with no increase in abscess or draining fistula count; HiSCR 75, a ≥75% reduction from baseline in HiSCR; HiSCR 90, a >90% reduction from baseline in HiSCR; IHS4-55, a ≥55% reduction in International Hidradenitis Suppurativa Score System; NRI, nonresponder imputation; PBO, placebo; UPA, upadacitinib.
^{a}Nonresponder imputation incorporating multiple imputation to handle missing data due to COVID (NRI-C).
*Nominal P = .087 vs in-trial placebo.
^{†}Nominal P < .001 vs in-trial placebo.
^{‡}Nominal P = .015 vs in-trial placebo.
^{§}Nominal P = .020 vs in-trial placebo.

### Subgroup Analyses

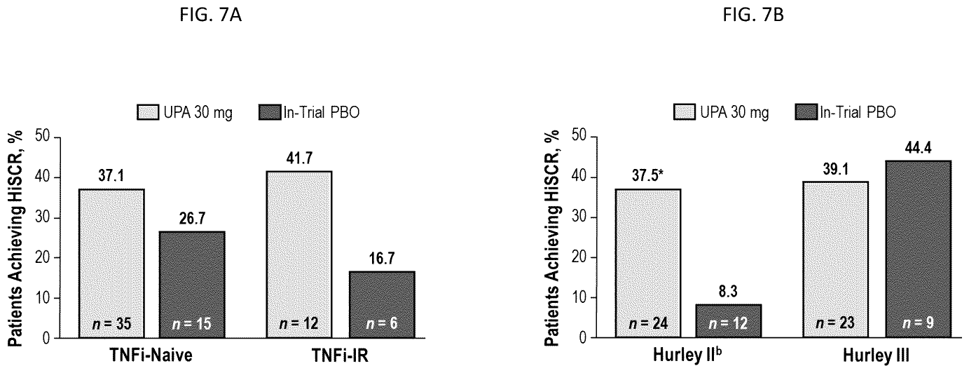

In a subgroup analysis, a higher proportion of patients receiving upadacitinib 30 mg achieved HiSCR compared with patients receiving in-trial placebo both among patients who previously had an inadequate response to TNF-α inhibitor therapy (upadacitinib, 41.7%; placebo, 16.7%; nominal P=0.115) and among patients naive to TNF-α inhibitor therapy (upadacitinib, 37.1%, placebo, 26.7%; nominal P=0.228) (FIG. 7A). HiSCR response rates with upadacitinib 30 mg were similar across Hurley stage subgroups and were similar to response rates observed in the overall population (FIG. 7B).

### Conclusion

Overall, this study met the primary endpoint showing that a statistically significantly greater proportion of patients with moderate-to-severe HS treated with upadacitinib 30 mg achieved HiSCR at week 12 compared with a prespecified historical placebo rate in adults by the primary analysis. The proportion of patients treated with upadacitinib 30 mg achieving Hi SCR was further improved or maintained through week 40, demonstrating the durability of response to upadacitinib. For patients who switched from placebo to upadacitinib 15 mg at week 12, a similar proportion of patients in the upadacitinib 15-mg group achieved Hi SCR at week 40 compared with those receiving upadacitinib 30 mg from baseline, although the study was not designed to compare the efficacy of upadacitinib 15 mg versus upadacitinib 30 mg. Findings from this Phase 2 study demonstrate that treatment with upadacitinib improved durable lesion and pain control in adults with moderate-to-severe HS. In summary, upadacitinib can provide higher levels of clinical efficacy across HS lesion types, including draining tunnels, to address the unmet needs of patients with moderate-to-severe HS.

1. A method for treating a human patient having moderate to severe hidradenitis suppurativa (HS), the method comprising orally administering once daily to the patient 30 mg of upadacitinib.

2. The method of claim 1, wherein the patient is an adult.

3. The method of claim 1, wherein the upadacitinib is administered to the patient for at least 12 weeks.

4. The method of claim 1, wherein a number of inflammatory lesions (AN count) is decreased by at least 50% at 12 weeks after the first daily administration, relative to an AN count prior to initiating treatment.

5. The method of claim 1, wherein a reduction in the Pain Numeric Rating Scale 30 (PNRS30) is achieved at 12 weeks after the first daily administration.

6. The method of claim 1, wherein the patient achieves a Hidradenitis Suppurativa Clinical Response 50 (HiSCR50) at 12 weeks after the first daily administration.

7. The method of claim 1, wherein the patient has HS lesions in at least two distinct anatomic areas prior to initiating treatment.

8. The method of claim 1, wherein the patient has had an inadequate response to or an intolerance to oral antibiotics.

9. The method of claim 1, wherein the patient failed to respond to or was intolerant of anti-TNF therapy.

10. The method of claim 1, wherein the patient has a total AN count equal to or greater than 5 prior to initiating the treating.

11. The method of claim 1, wherein the patient has a draining fistula count of less than or equal to 20 prior to initiating the treating.

12. The method of claim 1, wherein the patient experiences a reduction in flare through week 12, defined as an increase in AN count of at least 25% with a minimum increase of 2 relative to Baseline.

13. The method of claim 1, wherein one or more of the following is achieved at 12 weeks after the first daily administration, relative to that in a patient who has not received the treatment:
an improvement from Baseline in Dermatology Life Quality Index (DLQI);
an improvement from baseline in Hidradenitis Suppurativa Symptom Assessment (HSSA);
an improvement from baseline in HS-related swelling, assessed based on the HSSA;
an improvement from baseline in HS-related odor, assessed based on the HSSA;
an improvement from baseline in HS-related worst drainage, assessed based on the HSSA.

14. The method of claim 1, wherein a Hidradenitis Suppurativa Clinical Response 50 (HiSCR 50) is achieved at Week 16.

15. The method of claim 1, wherein a Hidradenitis Suppurativa Clinical Response 75 (HiSCR 75) is achieved at Week 16.

16. The method of claim 1, wherein a Hidradenitis Suppurativa Clinical Response 90 (HiSCR 90) is achieved at Week 16.

17. The method of claim 1, wherein a number of inflammatory lesions (AN count) is decreased in the patient relative to an AN count prior to initiating treatment.

18. The method of claim 1, wherein a draining fistula count is decreased in the patient relative to a draining fistula count prior to initiating treatment.

19. The method of claim 1, wherein a Numerical Rating Scale 30 (NRS30) is achieved in a Patient's Global Assessment of HS-related skin pain at Week 2, and wherein the patient had an NRS≥3 prior to initiating the treatment.

20. The method of claim 1, wherein a reduction in an experience of flare is achieved relative to that in a patient who has not received the treatment.

21. The method of claim 1, wherein at week 16, one or more of the following is achieved, relative to that in a patient who has not received the treatment:
an improvement from Baseline in Dermatology Life Quality Index (DLQI);
an improvement from Baseline in Hidradenitis Suppurativa Symptom Assessment (HSSA);
an improvement from Baseline in International Hidradenitis Suppurativa Severity Score System score;
an improvement from Baseline in HS-related odor, assessed based on the HSSA;
an improvement from Baseline in Hidradenitis Suppurativa Impact Assessment (HSIA).

22. The method of claim 1, wherein the patient achieves “much improved” or “very much improved” on the Total Patient Global Impression of Change (PGIC-Total).

23. The method of claim 1, wherein the patient achieves ≥1 grade improvement in Total Patient Global Impression of Severity relative to Baseline.

24. The method of claim 1, wherein the patient achieves an improvement from Baseline in Euro-QoL 5 Dimensions 5 Levels Health State (EQ-5D-5L).

25. The method of claim 1, wherein the patient achieves an improvement from Baseline in Work Productivity and Impairment (WPAI).

26. The method of claim 1, wherein the treatment provides a reduction in the incidence of disease progression.

27. The method of claim 1, wherein the treatment provides a reduction in cumulative analgesic use for HS-related skin pain relative to that in a patient who has not received the treatment at one or more of 16 weeks, 28 weeks, 36 weeks, 52 weeks, and 104 weeks.

28. The method of claim 1, comprising orally administering once daily to the patient an induction dose of 30 mg of upadacitinib for 16 weeks, followed by orally administering once daily to the patient a maintenance dose of 15 mg of upadacitinib.

29. A method for treating a human patient having moderate to severe hidradenitis suppurativa (HS), wherein the patient has failed to respond to or was intolerant of anti-TNF therapy, the method comprising orally administering once daily to the patient 30 mg of upadacitinib.

30. The method of claim 29, wherein the patient is at least 12 years of age.

31. The method of claim 29, wherein the patient is an adult.

32. The method of claim 29, wherein the method comprises orally administering upadacitinib to the patient daily for at least 16 weeks.

33. The method of claim 29, wherein a Hidradenitis Suppurativa Clinical Response 50 (HiSCR 50) is achieved at Week 16.

34. The method of claim 29, wherein a Hidradenitis Suppurativa Clinical Response 75 (HiSCR 75) is achieved at Week 16.

35. The method of claim 29, wherein a Hidradenitis Suppurativa Clinical Response 90 (HiSCR 90) is achieved at Week 16.

36. The method of claim 29, wherein a number of inflammatory lesions (AN count) is decreased in the patient relative to an AN count prior to initiating treatment.

37. The method of claim 29, wherein a draining fistula count is decreased in the patient relative to a draining fistula count prior to initiating treatment.

38. The method of claim 29, wherein a Numerical Rating Scale 30 (NRS30) is achieved in a Patient's Global Assessment of HS-related skin pain at Week 2, and wherein the patient had an NRS≥3 prior to initiating the treatment.

39. The method of claim 29, wherein a reduction in an experience of flare is achieved relative to that in a patient who has not received the treatment.

40. The method of claim 29, wherein at week 16, one or more of the following is achieved, relative to that in a patient who has not received the treatment:
an improvement from Baseline in Dermatology Life Quality Index (DLQI);
an improvement from Baseline in Hidradenitis Suppurativa Symptom Assessment (HSSA);
an improvement from Baseline in International Hidradenitis Suppurativa Severity Score System score;
an improvement from Baseline in HS-related odor, assessed based on the HSSA;
an improvement from Baseline in Hidradenitis Suppurativa Impact Assessment (HSIA).

41. The method of claim 29, wherein the patient achieves “much improved” or “very much improved” on the Total Patient Global Impression of Change (PGIC-Total).

42. The method of claim 29, wherein the patient achieves ≥1 grade improvement in Total Patient Global Impression of Severity relative to Baseline.

43. The method of claim 29, wherein the patient achieves an improvement from Baseline in Euro-QoL 5 Dimensions 5 Levels Health State (EQ-5D-5L).

44. The method of claim 29, wherein the patient achieves an improvement from Baseline in Work Productivity and Impairment (WPAI).

45. The method of claim 29, wherein the treatment provides a reduction in the incidence of disease progression.

46. The method of claim 29, wherein the treatment provides a reduction in cumulative analgesic use for HS-related skin pain relative to that in a patient who has not received the treatment at one or more of 16 weeks, 28 weeks, 36 weeks, 52 weeks, and 104 weeks.

47. The method of claim 29, comprising orally administering once daily to the patient an induction dose of 30 mg of upadacitinib for 16 weeks, followed by orally administering once daily to the patient a maintenance dose of 15 mg of upadacitinib.
